(19) United States
(12) Patent Application Publication
(10) Pub. No.: US 2008/0226697 A1
(43) Pub. Date: Sep. 18, 2008

(54) Patch for External Use with Elevated Content of Absorption Promoter in Pressure-Sensitive Adhesive Base

(75) Inventors: Toshiro Yamaguchi, Ibaraki (JP); Tsuyoshi Endo, Ibaraki (JP); Tetsuro Tateishi, Ibaraki (JP); Naruhito Higo, Ibaraki (JP)

Correspondence Address: LICATA & TYRRELL P.C., 66 E. MAIN STREET, MARLTON, NJ, 08053, US

(73) Assignee: HISAMITSU PHARMACEUTICAL CO., INC., Saga (JP)

(21) Appl. No.: 11/578,892

(22) PCT Filed: Apr. 21, 2005
(86) PCT No.: PCT/JP05/07647

§ 371 Date: Dec. 11, 2006

(30) Foreign Application Priority Data
Apr. 21, 2004 (JP) 2004-125797

(51) Int. Cl. A61K 9/70; A61K 31/216

(52) U.S. Cl. 424/448; 514/534

(57) ABSTRACT

A pressure-sensitive adhesive composition for transdermal absorption which contains a drug and a transdermal absorption promoter for the drug, characterized by further containing polyvinylpyrrolidone so as to stabilize the transdermal absorption promoter contained in the adhesive composition. Thus, the transdermal absorption promoter can be prevented from vaporization or degradation and a patch for external use containing the transdermal absorption promoter at a high concentration in using can be provided.

### DETAILED DESCRIPTION OF THE INVENTION

1. Technical Field

The invention relates to a pressure-sensitive adhesive composition for transdermal absorption and a patch for external use which have elevated content of an absorption promoter in a pressure-sensitive adhesive base.

2. Background Art

Conventionally, as administration methods for drugs, various methods such as an oral administration, a rectal administration, an intracutaneous administration and an intravenous administration have been known, though the oral administration has widely been adopted. However, in the oral administration, there are drawbacks that a drug is susceptible to a first-pass effect in the liver after absorption of the drug and an unnecessary high blood concentration is recognized for a while after the administration, etc. Additionally, in the oral administration, many side effects such as a gastrointestinal tract disorder, a vomiting feeling and a loss of appetite have been reported. Further, in an aging society of recent years, when considering that patients with a reduced swallowing power increase, preparations easy for administration are desired clinically. Therefore, as a preparation wherein these drawbacks in the oral administration are dissolved and patients can easily take it with more safety and persistence, the development of a patch for external use has actively been carried out, and the products are commercially available.

However, owing to low transdermal absorption in many drugs, the development of patches for external use is difficult, and the patches for external use have not sufficiently attained the object. Namely, since a normal skin has a barrier function to protect the penetration of foreign substances, in a base used in a usual patch for external use a sufficient transdermal absorption of a drug is hardly attained in many cases.

Therefore, a device to increase the transdermal absorption of a drug through a stratum corneum of the skin is needed, and a method to mix a transdermal absorption promoter, for example, such as an organic acid in a base is known.

For example, a patch containing a salt of a nonsteroidal anti-inflammatory agent, a transdermal absorption promoter and glycol (JP, A, 62-181226) or a patch containing a nonsteroidal anti-inflammatory agent in a salt form of an alkaline metal and an organic acid stronger in acidity than the nonsteroidal anti-inflammatory agent in the free state (JP, B, 7-47535) are reported.

Further, a patch for external use is reported in which an ion-pair is formed by letting not an organic acid alone but the organic acid and its salt together as a transdermal absorption promoter be contained in a pressure-sensitive adhesive composition and a skin permeability of a drug is improved compared with use of the organic acid alone (WO01/007018 A1).

However, in a conventional method, due to a cause that a transdermal absorption promoter of a drug was volatile or unstable, the content of the transdermal absorption promoter diminished when using a patch compared with the preparation amount, and therefore, there was a problem that a sufficient transdermal absorption effect could not be obtained. In addition, in case of increasing the mix concentration of acetic acid in anticipation of the volatility or the degradation, the ooze of a transdermal absorption promoter from a base occurred, and it was unsuitable for a preparation.

In the meantime, use of polyvinylpyrrolidone has been known as a solubility enhancer of a drug in a pressure-sensitive adhesive agent, and it is reported that polyvinylpyrrolidone does not substantially reduce a permeation rate of a drug or adhesiveness of a composition but can inhibit crystallization of the drug and dissolve it (JP, A, 9-511987). However, in the publication, a combined use of an absorption promoter of a drug is not disclosed.

### DISCLOSURE OF THE INVENTION

### Problems to Be Solved by the Invention

Consequently, the problem to be solved by the invention is to provide a patch for external use which inhibits the volatility or degradation of a transdermal absorption promoter by stabilization of the transdermal absorption promoter contained in a pressure-sensitive adhesive composition and contains the absorption promoter at a high concentration in using.

### Means for Solving the Problems

The inventors made extensive researches to solve the above problems and found that the vaporization or degradation of a transdermal absorption promoter can be prevented by containing polyvinylpyrrolidone in a pressure-sensitive adhesive composition containing the transdermal absorption promoter, resulting an unexpected increase of the skin absorbability, and accomplished the invention as a result of further investigation.

Namely, the invention relates to a pressure-sensitive adhesive composition for transdermal absorption, which contains a drug and a transdermal absorption promoter for the drug, wherein polyvinylpyrrolidone is further contained.

In addition, the invention relates to the pressure-sensitive adhesive composition for transdermal absorption, wherein the transdermal absorption promoter is volatile or degradable.

Further, the invention relates to the pressure-sensitive adhesive composition for transdermal absorption, wherein the transdermal absorption promoter is an organic acid.

In addition, the invention relates to the pressure-sensitive adhesive composition for transdermal absorption, wherein the transdermal absorption promoter is acetic acid.

Further, the invention relates to the pressure-sensitive adhesive composition for transdermal absorption, which further contains an organic acid salt.

In addition, the invention relates to the pressure-sensitive adhesive composition for transdermal absorption, wherein the organic acid salt is sodium acetate. Further, the invention relates to the pressure-sensitive adhesive composition for transdermal absorption, wherein the transdermal absorption promoter is acetic acid and the organic acid salt is sodium acetate.

In addition, the invention relates to the pressure-sensitive adhesive composition for transdermal absorption, wherein the drug is oxybutynin.

Further, the invention relates to the pressure-sensitive adhesive composition for transdermal absorption, which further contains an acrylic polymer.

In addition, the invention relates to the pressure-sensitive adhesive composition for transdermal absorption, wherein the acrylic polymer is copolymer containing acrylate having at least —OH group and vinyl acetate.

Further, the invention relates to a patch for external use, which contains the above pressure-sensitive adhesive composition for transdermal absorption.

Conventionally, although polyvinylpyrrolidone was known as a solubility enhancer of a drug or a thickener, it was surprisingly found for the first time in the invention that it could contribute to stabilization of an absorption promoter of a drug. And, the invention exerts a particular effect to absorbability of the drug.

### EFFECT OF THE INVENTION

The pressure-sensitive adhesive composition for transdermal absorption of the invention contains a transdermal absorption promoter at a high concentration in the pressure-sensitive adhesive composition so as to be stabilized by mixing polyvinylpyrrolidone in the pressure-sensitive adhesive composition, and can improve a transdermal absorption effect of a drug. Additionally, the patch for external use of the invention can improve the transdermal absorption effect of the drug without having an adverse effect to the adhesiveness and stability of the pressure-sensitive adhesive composition.

### BEST MODE FOR CARRYING OUT THE INVENTION

In the following, the invention is explained in detail.

The patch for external use of the invention may contain a backing layer supporting a pressure-sensitive adhesive layer and a removable paper layer set on the pressure-sensitive adhesive layer. In the patch for external use of the invention, a drug, a transdermal absorption promoter of the drug and polyvinylpyrrolidone are contained in the above pressure-sensitive adhesive layer.

A drug contained in the pressure-sensitive adhesive composition of the invention is not limited particularly if it is used generally. Examples include hypnotic-sedative agents (flurazepam hydrochloride, rilmazafone hydrochloride, phenobarbital, amobarbital, etc.), antipyretic-antiinflammatory-analgesic agents (butorphanol tartarate, perisoxal citrate, acetaminophen, mefenamic acid, diclofenac sodium, aspirin, alclofenac, ketoprofen, flurbiprofen, naproxen, piroxicam, pentazocine, indometacin, glycol salicylate, aminopyrine, loxoprofen, etc.), steroidal anti-inflammatory agents (hydrocortisone, predonisolone, dexamethasone, betamethasone, etc.), excitation-analeptic agents (methamphetamine hydrochloride, methylphenidate hydrochloride, etc.), psychotropic agents (imipramine hydrochloride, diazepam, celtralin hydrochloride, fluvoxamine maleate, paroxetine hydrochloride, citalopram hydrobromide, fluoxetine hydrochloride, alprazolam, chlorpromazine hydrochloride, etc.), hormones (estradiol, estriol, progesterone, norethisterone acetate, metenolone acetate, testosterone, etc.), local anesthetic agents (lidocain hydrochloride, procaine hydrochloride, tetracaine hydrochloride, etc.), agents for urinary organs (oxybutynin, oxybutynin hydrochloride, tamusulosin hydrochloride, etc.), skeletalmuscle relaxants (tizanidine hydrochloride, eperisone hydrochloride, pridinol mesilate, etc.), autonomic agents (carpronium chloride, neostigmine bromide, etc.), antiepileptic agents (sodium valproate, clonazepam, etc.), anti-Parkinson's disease agents (pergolide mesilate, bromocriptine mesilate, trihexyphenidyl hydrochloride, amantadine hydrochloride, ropinirole hydrochloride, carbergoline, etc.), antihistaminic agents (clemastine fumarate, diphenhydramine tannate, etc.), diuretic agents (hydrofulmetiazide, furosemide, etc.), respiratory stimulants (lobeline hydrochloride, dimorpholamine, naloxone hydrochloride, etc.), anti-migraine agents (dihydroergotamine mesilate, sumatriptan, etc.), bronchodilator agents (tulobuterol hydrochloride, procaterol hydrochloride, etc.), cardiotonic agents (isoprenaline hydrochloride, dopamine hydrochloride, etc.), coronary dilators (diltiazem hydrochloride, verapamil hydrochloride, isosorbide nitrate, nitroglycerin, etc.), peripheral vasodilators (nicametate citrate, tolazoline hydrochloride, etc.), smoking cessation aid agents (nicotine, etc.), cardiovascular agents (flunarizine hydrochloride, nicardipine hydrochloride, benidipine hydrochloride, efonidipine hydrochloride, bisoprolol fumarate, metoprolol tartrate, etc.), antiarrhythmic agents (propranolol hydrochloride, alprenolol hydrochloride, nadolol, etc.), antiallergic agents (ketotifen fumarate, azelastine hydrochloride, etc.), anti-dizziness agents (betahistine mesilate, difenidol hydrochloride, etc.), serotonin receptor antagonistic agents (ondansetron hydrochloride, granisetron hydrochloride, etc.), gastrointestinal motility improving agents (domperidone, cisapride, etc.), blood glucose lowering agents (glibenclamide, tolbutamide, etc.), anorectic agents (mazindol, etc.), chemotherapeutic agents (isoniazid, ethionamide, etc.), anticoagulants (warfarin potassium, etc.), agents for anti-Alzheimer disease (tacrine, donepezil hydrochloride, etc.), antipodagric agents (colchicine, probenecid, etc.), narcotic analgesic agents (morphine sulfate, fentanyl citrate, etc.), though a basic drug is preferable, and oxybutynin is particularly preferable.

These drugs may be used alone in one kind or in a combination of two or more kinds. In addition, the mix amount of these drugs is preferably 1-40 mass % based on the mass of the total composition of the pressure-sensitive adhesive layer by consideration of a sufficient permeation amount as the patch for external use and a drug efficacy, a pressure-sensitive adhesive physical property, etc.

The transdermal absorption promoter contained in the pressure-sensitive adhesive composition of the invention is not particularly limited if the absorption promoting effect is known. Examples includes C₂-C₇ carboxylic acids, C₆-C₂₀ fatty acids, fatty alcohols, fatty acid esters or ethers, aromatic organic acids, aromatic alcohols, aromatic organic acid esters or ethers (these may be saturated or unsaturated, and may be either cyclic, straight or branched), furthermore, lactates, acetates, monoterpene compounds, sesquiterpene compounds, Azone, Azone derivatives, glycerol fatty acid esters, sorbitan fatty acid esters (Span type), polysorbates (Tween type), polyethylene glycol fatty acid esters, polyoxyethylene hardened castor oils (HCO type), sucrose fatty acid esters, and the like, preferably organic acids, in particular C₂-C₇ carboxylic acids, more preferably aliphatic (mono-, di-, tri-)carboxylic acids (e.g., acetic acid, propionic acid, isobutyric acid, lactic acid, maleic acid, fumaric acid, pyruvic acid, oxalicacid, succinic acid, tartaric acid, etc.), aromatic carboxylic acids (e.g., salicylic acid, benzoic acid, etc.). Among these, acetic acid is particularly preferable.

Here, the transdermal absorption promoters which are volatile or degradable have boiling points not more than 165° C., and specifically, examples includes acetic acid, propionic acid, butyric acid, etc.

These transdermal absorption promoters may be used alone in one kind or in a combination of two kinds. In addition, the mix amount of these transdermal absorption promoters is preferably 1-20 mass % based on the mass of the total composition of the pressure-sensitive adhesive layer by consideration of stability as a patch for external use, transdermal absorption of a drug and irritation to the skin, more preferably 2-15 mass %, in particular preferably 3-10 mass %.

In the pressure-sensitive adhesive composition of the patch for external use of the invention, the transdermal absorption promoter is preferably contained not less than one equivalent mole against a drug when used. Therefore, the mix amount of polyvinyl pyrrolidone contained in the pressure-sensitive adhesive composition of the invention is preferably 1-40 mass % based on the mass of the total composition of the pressure-sensitive adhesive layer considering concentration of the transdermal absorption promoter and physical properties, more preferably 2-30 mass %, in particular preferably 3-20 mass %.

An organic acid salt which can be used in the pressure-sensitive adhesive layer of the patch for external use of the invention is not particularly limited. Examples includes aqueous inorganic salts of each of aliphatic (mono-, di-, tri-) carboxylic acids (e.g., acetic acid, propionic acid, isobutylic acid, caproic acid, caprylic acid, lactic acid, maleic acid, pyruvic acid, oxalic acid, succinic acid, tartaric acid, etc.), aromatic carboxylic acids (e.g., phthalic acid, salicylic acid, benzoic acid, acetyl salicylate, etc.), alkyl sulfonic acids (e.g., ethanesulfonic acid, propyl sulfonic acid, butanesulfonic acid, polyoxyethylene alkyl ether sulfonic acid, etc.), alkyl sulfonic acid derivatives (e.g., N-2-hydroxyethylpiperidine-N′-2-ethanesulfonic acid (hereinafter abbreviated as HEPES), etc.) and cholic acid derivatives (e.g., dehydrocholic acid, etc.). Among these, metal salts of carboxylic acids are preferable, and sodium acetate is in particular preferable. In addition, although these organic acid salts may be anhydrous or hydrates, the anhydrous are preferable in case of using them in a hydrophobic pressure-sensitive adhesive layer.

These organic acid salts may be used alone in one kind or in a combination of two or more kinds. In addition, considering physical properties, a sufficient permeation amount as the patch for external use and irritation to the skin, the mix amount of these organic acid salts is preferably 1-20 mass % based on the mass of the total composition of the pressure-sensitive adhesive layer, more preferably 2-10 mass %, in particular preferably 3-7 mass %.

Although the combination of a transdermal absorption promoter and an organic acid salt which are contained in the pressure-sensitive adhesive composition of the invention may be any combination of the above transdermal absorption promoter and organic acid salt, preferably the combination of acetic acid and sodium acetate.

An acrylic polymer which can be used in the pressure-sensitive adhesive composition of the invention is not particularly limited. Examples includes copolymer of 2-ethylhexyl acrylate/vinyl acetate copolymer, copolymer of 2-ethylhexyl acrylate/hydroxyethyl acrylate/vinyl acetate, copolymer of 2-ethylhexyl acrylate/hydroxyethyl acrylate/acrylic acid/vinyl acetate, copolymer of 2-ethylhexyl acrylate/methyl acrylate/acrylic acid, copolymer of 2-ethylhexyl acrylate/methyl acrylate/acrylic acid/vinyl acetate, copolymer of 2-ethylhexyl acrylate/vinylpyrrolidone/hydroxyethyl acrylate/acrylic acid/vinyl acetate, copolymer of 2-ethylhexyl acrylate/vinylpyrrolidone/hydroxyethyl acrylate/vinyl acetate, and copolymer of 2-ethylhexyl acrylate/N-vinyl-2-pyrrolidone/1,6-hehaneglycol dimethacrylate, and in particular, copolymer containing acrylate having at least one —OH group and vinyl acetate is preferable. The mix amount of the acrylic polymer is preferably 30-95 mass % based on the mass of the total composition of the pressure-sensitive adhesive layer considering formation of the pressure-sensitive adhesive layer and a sufficient permeability, more preferably 40-80 mass %, in particular preferably 50-70 mass %.

In addition, the mix ratio between the transdermal absorption promoter and polyvinylpyrrolidone is preferably 1:10-5:1 (mass ratio) considering permeability to the skin, more preferably 1:5-3:1 (mass ratio), in particular preferably 1:3-2:1 (mass ratio).

Further, the mix ratio between the transdermal absorption promoter and the drug is preferably 10:1-1:5 (equivalent ratio) considering permeability to the skin, more preferably 8:1-1:2 (equivalent ratio), in particular preferably 6:1-1:2 (equivalent ratio).

In addition, the mix ratio between polyvinylpyrrolidone and the drug is preferably 1:10-1:10 (mass ratio) considering permeability to the skin, more preferably 1:5-5:1 (mass ratio), in particular preferably 1:3-3:1 (mass ratio).

In addition to the above drug, transdermal absorption promoter, polyvinylpyrrolidone, organic acid salt and acrylic polymer, the pressure-sensitive adhesive layer of the patch for external use of the invention may contain a plasticizer, a tackifying resin, and as required, the other additive, etc.

As a plasticizer which can be used in the pressure-sensitive adhesive layer of the patch for external use of the invention, examples include petroleum oils (e.g., paraffin type process oil, naphthene type process oil, aromatic type process oil, etc.), squalane, squalene, vegetable oils (e.g., olive oil, camellia oil, castor oil, tall oil, peanut oil), silicone oil, dibasic acid esters (e.g., dibutyl phthalate, dioctyl phthalate, etc.), liquid rubber (e.g., polybutene, liquid isoprene rubber), diethylene glycol, polyethylene glycol, glycol salicylate, propylene glycol, dipropylene glycol, triacetin, triethyl citrate, crotamiton, diethyl sebacate, etc. Among these, liquid paraffin, liquid polybutene, glycol salicylate and crotamiton are in particular preferable.

These plasticizers may be used alone in one kind or in a combination of two kinds. In addition, considering sufficient permeability and maintenance of sufficient cohesiveness as a patch preparation, the mix amount is preferably 10-70 mass % in total, more preferably 10-60 mass %, in particular preferably 10-50 mass %.

As a tackifying resin which can be used in the pressure-sensitive adhesive layer of the patch for external use of the invention, examples include rosin derivatives (e.g., rosin, glycerol esters of rosin, hydrogenated rosin, glycerol esters of hydrogenated rosin, pentaerythritol esters of rosin, etc.), alicyclic saturated hydrocarbon resins, aliphatic hydrocarbon resins, terpene resins, maleic acid resins, etc. Among these, glycerol esters of hydrogenated rosin, alicyclic saturated hydrocarbon resins, aliphatic hydrocarbon resins and terpene resins are in particular preferable.

These tackifying resins may be used alone in one kind or in a combination of two or more kinds. In addition, considering sufficient adhesive strength as a patch preparation and irritation to the skin at the time of removal, the mix amount of the tackifying resins is preferably 10-70 mass % based on the mass of the total composition of the pressure-sensitive adhesive layer, more preferably 15-60 mass %, in particular preferably 20-50 mass %.

Further, as required, additives such as an antioxidant, filler, cross-linking agent, preservative and ultraviolet absorber can be used in the pressure-sensitive adhesive layer of the patch for external use of the invention.

As antioxidants, tocopherol and its ester derivatives, ascorbic acid, ascorbic acid-stearic acid ester, nordihydroguaretic acid, dibutyl hydroxyl toluene (BHT), butyl hydroxyanisole, and the like can be used.

As fillers, calcium carbonate, magnesium carbonate, silicates (e.g., aluminum silicate, magnesium silicate, etc.), silicic acid, barium sulfate, calcium sulfate, calcium zincate, zinc oxide, titanium oxide and the like can be used.

As cross-linking agents, thermosetting resins such as amino resins, phenol resins, epoxy resins, alkyd resins and unsaturated polyesters, isocyanate compounds, block isocyanate compounds, organic cross-linking agents, and inorganic cross-linking agents such as metals or metal compounds can be used.

As preservatives, ethyl p-hydroxybenzoate, propyl p-hydroxybenzoate, butyl p-hydroxybenzoate and the like can be used.

As ultraviolet absorbers, p-aminobenzoic acid derivatives, anthranilic acid derivatives, salicylic acid derivatives, coumarin derivatives, amino acid compounds, imidazoline derivatives, pyrimidine derivatives, dioxane derivatives and the like can be used.

The mix amount of additives such as an antioxidant, filler, cross-linking agent, preservative and ultraviolet absorber is preferably not more than 10 mass % in total based on the mass of the total composition of the pressure-sensitive adhesive layer of the patch for external use of the invention, more preferably not more than 5 mass %, in particular preferably not more than 2 mass %.

The preparation method of the patch for external use of the invention having such composition is not limited, and it can be prepared by any method. For example, a base composition containing a drug is heat-melted, coated on a removable paper or a backing, followed by affixing it to the backing or the removable paper to give the patch for external use. In addition, base ingredients containing a drug are dissolved in solvent such as toluene, hexane, ethyl acetate, methanol or ethanol, spread on a removable paper or a backing, dried to remove solvent, followed by affixing it to the backing or the removable paper to give the patch for external use.

The patch for external use of the invention is preferably a non-aqueous patch for external use. Further, as to the patch for external use of the invention, other constituent and materials for each constituent part may be any type, if it contains the drug, transdermal absorption promoter of the drug and polyvinylpyrrolidone as described above.

The backing layer which can be set up to support the pressure-sensitive adhesive layer can be formed using an elastic or non-elastic backing. As the backing, for example, fabric, non-woven fabric, polyurethane, polyester, polyvinyl acetate, polyvinylidene chloride, polyethylene, polyethylene terephthalate, aluminum sheet and the like, or composite materials thereof can be used by selection.

In addition, as to removable paper layer which can be set on the pressure-sensitive adhesive layer, for example, films of polyethylene terephthalate, polyester, polyvinyl chloride, polyvinylidene chloride or the like which surface to be contacted with the pressure-sensitive adhesive layer is treated with silicone, or laminate films of a high-quality paper or the like and polyolefine can be used by selection.

### EXAMPLE

In the following, the invention is specifically explained in more detail by the examples. Further, in the comparative example and the examples, all % mean mass %.

### Example 1

### Preparation of Patch

Ethanol was added to glacial acetic acid, oxybutynin, polyvinylpyrrolidone (K90) and sodium acetate which was ground beforehand and mixed thoroughly. To the mixture was added the acrylic pressure-sensitive agent (Duro-Tak 87-2516, manufactured by National Starch & Chemicals Co., Ltd.) to prepare a coating liquid having the composition shown in the following.

Composition:

| | |
|---|---|
| Acrylic polymer | 66.6% |
| Polyvinylpyrrolidone (K90) | 5.0% |
| Sodium acetate | 3.4% |
| Glacial acetic acid | 10.0% |
| Oxybutynin | 15% |

Then, the obtained coating liquid was coated on a removable paper made of polyethylene terephthalate which was treated with silicone, dried to remove solvent to form a membrane of a pressure-sensitive adhesive layer which was affixed to a polyethylene terephthalate side of a laminate backing of polyethylene terephthalate and copolymer of ethylene/vinyl acetate to obtain an aimed patch.

### (Skin Permeability Test)

The skin permeability test was carried out in the following procedure using the obtained patch.

First, a back part skin of a hairless mouse was stripped, and the dermal side was placed to a receptor layer side and installed in a flow-through cell in which warm water of 32° C. was circulated around the outer part. Then, the patch (application area of the preparation, 5 cm²) obtained in the example 1 was applied on the stratum corneum side, and samplings were carried out at every two hour for 20 hours at 5 ml/hour (hr) using the physiological saline in the receptor layer. The flow amount was measured, and the drug level was also measured by a high-performance liquid chromatography. Based on the actual value, the drug permeation rate per hour was calculated, and the drug permeation rate per unit area of the skin at a steady state was obtained. The obtained results are shown in Table 1.

### (Acetic Acid Determination)

The prepared preparation was punched into 10 cm², put in tetrahydrofuran 10 ml and shaken for 1 hr. From this, 4 ml of the mixture was taken, filtered, then added with an internal standard substance (21.5 mmol/L methanolic fumaric acid solution) 5 ml and methanol 20 ml, increased to 100 ml with water and quantitated by a high-performance liquid chromatography.

### Example 2

Except the concentration of polyvinylpyrrolidone (K90) in the example 1, the coating liquid having the composition shown in the following was prepared in the same way as the example 1.

Composition:

| | |
|---|---|
| Acrylic polymer | 51.6% |
| Polyvinylpyrrolidone (K90) | 20.0% |
| Sodium acetate | 3.4% |
| Glacial acetic acid | 10.0% |
| Oxybutynin | 15% |

Then, the obtained coating liquid was coated on a removable paper made of polyethylene terephthalate which was treated with silicone, dried to remove solvent to form a membrane of a pressure-sensitive adhesive layer which was affixed to a polyethylene terephthalate side of a laminate backing of polyethylene terephthalate and copolymer of ethylene/vinyl acetate to obtain an aimed patch.

As for the obtained patch, the skin permeability test and the acetic acid determination were carried out in the same way as the example 1, and the results are shown in Table 1.

### Example 3

Except the concentration of sodium acetate in the example 2, the coating liquid having the composition shown in the following was prepared in the same way as the example 2.

Composition:

| | |
|---|---|
| Acrylic polymer | 49.8% |
| Polyvinylpyrrolidone (K90) | 20.0% |
| Sodium acetate | 5.2% |
| Glacial acetic acid | 10.0% |
| Oxybutynin | 15% |

Then, the obtained coating liquid was coated on a removable paper made of polyethylene terephthalate which was treated with silicone, dried to remove solvent to form a membrane of a pressure-sensitive adhesive layer which was affixed to a polyethylene terephthalate side of a laminate backing of polyethylene terephthalate and copolymer of ethylene/vinyl acetate to obtain an aimed patch.

As for the obtained patch, the skin permeability test and the acetic acid determination were carried out in the same way as the example 1, and the results are shown in Table 1.

### Example 4

Except the concentration of sodium acetate in the example 3, the coating liquid having the composition shown in the following was prepared in the same way as the example 3.

Composition:

| | |
|---|---|
| Acrylic polymer | 48.1% |
| Polyvinylpyrrolidone (K90) | 20.0% |
| Sodium acetate | 6.9% |
| Glacial acetic acid | 10.0% |
| Oxybutynin | 15% |

Then, the obtained coating liquid was coated on a removable paper made of polyethylene terephthalate which was treated with silicone, dried to remove solvent to form a membrane of a pressure-sensitive adhesive layer which was affixed to a polyethylene terephthalate side of a laminate backing of polyethylene terephthalate and copolymer of ethylene/vinyl acetate to obtain an aimed patch.

As for the obtained patch, the skin permeability test and the acetic acid determination were carried out in the same way as the example 1, and the results are shown in Table 1.

### Comparative Example 1

Except no use of polyvinylpyrrolidone and different concentration of sodium acetate in the example 1, the coating liquid having the composition shown in the following was prepared in the same way as the example 1.

Composition:

| | |
|---|---|
| Acrylic polymer | 71.2% |
| Sodium acetate | 3.8% |
| Glacial acetic acid | 10.0% |
| Oxybutynin | 15% |

Then, the obtained coating liquid was coated on a removable paper made of polyethylene terephthalate which was treated with silicone, dried to remove solvent to form a membrane of a pressure-sensitive adhesive layer which was affixed to a polyethylene terephthalate side of a laminate backing of polyethylene terephthalate and copolymer of ethylene/vinyl acetate to obtain an aimed patch.

As for the obtained patch, the skin permeability test and the acetic acid determination were carried out in the same way as the example 1, and the results are shown in Table 1.

### [Table 1]

TABLE 1

| | | | | | | AcOH | |
|---|---|---|---|---|---|---|---|
| | | | | | | (Actual | |
| | Pressure- | | | | | value) | |
| | sensitive | | | | | AcOH | |
| | adhesive | | | Absorption | | (vs. Drug) | |
| | agent | | PVP | promoter | | Equivalent | Flux |
| Lot No. | Duro-Tak | Drug % | K90 % | AcOH % | AcONa % | mole | μg/cm²/hr |
| Comparative | 87-2516 | 15 | — | 10 | 3.8 | 0.95 | 9.27 |
| example | | | | | | | |
| Example 1 | 87-2516 | 15 | 5 | 10 | 3.4 | 1.59 | 13.21 |
| Example 2 | 87-2516 | 15 | 20 | 10 | 3.4 | 1.58 | 12.67 |
| Example 3 | 87-2516 | 15 | 20 | 10 | 5.2 | 2.62 | 14.65 |
| Example 4 | 87-2516 | 15 | 20 | 10 | 6.9 | 2.97 | 15.52 |

### INDUSTRIAL APPLICABILITY

The pressure-sensitive adhesive composition for transdermal absorption of the invention is excellent in a transdermal absorption effect of a drug, and therefore, can be used as a patch for external use to the skin and greatly contributes to development of related industries.

1. A pressure-sensitive adhesive composition for transdermal absorption, which contains a drug and a transdermal absorption promoter for the drug, wherein polyvinylpyrrolidone is further contained.

2. The pressure-sensitive adhesive composition for transdermal absorption according to claim 1, wherein the transdermal absorption promoter is volatile or degradable.

3. The pressure-sensitive adhesive composition for transdermal absorption according to claim 1, wherein the transdermal absorption promoter is an organic acid.

4. The pressure-sensitive adhesive composition for transdermal absorption according to claim 3, wherein the transdermal absorption promoter is acetic acid.

5. The pressure-sensitive adhesive composition for transdermal absorption according to claim 1, which further contains an organic acid salt.

6. The pressure-sensitive adhesive composition for transdermal absorption according to claim 5, wherein the organic acid salt is sodium acetate.

7. The pressure-sensitive adhesive composition for transdermal absorption according to claim 6, wherein the transdermal absorption promoter is acetic acid and the organic acid salt is sodium acetate.

8. The pressure-sensitive adhesive composition for transdermal absorption according to claim 1, wherein the drug is oxybutynin.

9. The pressure-sensitive adhesive composition for transdermal absorption according to claim 1, which further contains an acrylic polymer.

10. The pressure-sensitive adhesive composition for transdermal absorption according to claim 9, wherein the acrylic polymer is copolymer containing acrylate having at least —OH group and vinyl acetate.

11. A patch for external use, which contains the pressure-sensitive adhesive composition for transdermal absorption according to claim 1.
